(19) United States
(12) Patent Application Publication
(10) Pub. No.: US 2023/0187030 A1
(43) Pub. Date: Jun. 15, 2023

(54) RAPID QUANTITATIVE EVALUATION METHOD FOR TASTE CHARACTERISTICS OF FRIED RICE

(71) Applicant: Jiangsu University, Jiangsu (CN)

(72) Inventors: Jiyong SHI, Jiangsu (CN); Xiaobo ZOU, Jiangsu (CN); Xiaowei HUANG, Jiangsu (CN); Zhihua LI, Jiangsu (CN); Tingting SHEN, Jiangsu (CN); Xin LI, Jiangsu (CN); Pengjing CUI, Jiangsu (CN); Jianbo XIAO, Jiangsu (CN); Xinai ZHANG, Jiangsu (CN); Di ZHANG, Jiangsu (CN); Chenguang ZHOU, Jiangsu (CN); Yang ZHANG, Jiangsu (CN); Mengxue LIU, Jiangsu (CN)

(73) Assignee: Jiangsu University, Jiangsu (CN)

(21) Appl. No.: 17/907,963

(22) PCT Filed: Dec. 7, 2021
(86) PCT No.: PCT/CN2021/135904

§ 371 Date: Aug. 29, 2022

(30) Foreign Application Priority Data
Nov. 24, 2021 (CN) 202111403368.9

(51) Int. Cl. G16C 20/30; G16C 20/70

(52) U.S. Cl. CPC G16C 20/30; G16C 20/70

(57) ABSTRACT

The present disclosure belongs to the technical field of food processing, and in particular relates to a rapid and non-destructive quantitative evaluation method for taste characteristics of fried rice. The method includes three steps: constructing a quantitative model of seasoning for the fried rice, constructing a model for identifying types of materials of the fried rice, and performing quantitative characterization of the taste characteristics of the fried rice. The quantitative model of the seasoning is established based on sensitivity of a spectral signal to changes of the content of seasoning liquid. An analytic equation of key parameters of the quantitative model of the seasoning is established according to characteristics that the total amount of the seasoning in finished fried rice is equal to the total amount of the seasoning added during frying of the fried rice. The quantitative model of the seasoning is rapidly constructed by solving the equations.

### TECHNICAL FIELD

The present disclosure belongs to the technical field of food processing, and in particular relates to a rapid and non-destructive quantitative evaluation method for taste characteristics of fried rice.

### BACKGROUND

Fried rice is a kind of delicacy cooked with rice, side dishes, and seasonings, and it is nutritious and delicious, diverse in styles, and convenient to make. The common cooking process of fried rice involves frying rice of a certain degree of maturity with different kinds of side dishes, and adding different flavors of seasonings for seasoning at the same time. High-quality fried rice and special fried rice have higher requirements in color, aroma, taste, and appearance. Taste has the greatest impact on consumers' feelings when they taste the fried rice, and it is the key to determining the quality of the fried rice and endowing it with characteristics. However, fried rice not only has complex ingredients, but also has a small granularity and diverse shapes. Therefore, how to quantitatively detect the taste characteristics of fried rice is the key to judging and making high-quality fried rice.

The existing methods for evaluating the taste characteristics include an artificial sensory method, a physical and chemical analysis method, and a non-destructive testing method.

The artificial sensory method mainly uses human senses to perceive product characteristics or properties, which can realize the evaluation of the taste characteristics. In terms of evaluation of the taste characteristics by the artificial sensory method, the disclosure patent CN112986506A discloses a method for evaluating taste quality of rice using sensory. However, the artificial sensory method has shortcomings such as strong subjectivity and low detection accuracy, and it is difficult to evaluate the taste characteristics of the fried rice objectively and accurately. The physical and chemical analysis method is to perform qualitative and quantitative analysis on the taste and flavor-related ingredients of food by physical and chemical analysis means, so as to determine the taste characteristics of the food (such as patent CN113138257A). However, the physical and chemical analysis method has high detection cost, time-consuming detection process, and high requirements for operators. It is difficult to realize fast and online detection of the taste characteristics of fried rice. The non-destructive testing method can establish a qualitative and quantitative detection model of food flavor components using the correlation between a non-destructive testing signal such as photoelectricity and the taste characteristic components of food without destroying the original state of the sample, so as to realize non-destructive and rapid detection of the taste characteristics of food, such as methods for evaluating the taste characteristics based on the spectroscopy (CN111007040A) and electrochemical method (CN108037256B). However, the existing non-destructive testing methods are mainly used for the detection of the content of the taste characteristic ingredients of samples, and it is difficult to accurately analyze the taste ability of different food ingredients and the distribution of flavoring components in complex foods such as the fried rice. At the same time, a modeling process corresponding to the conventional non-destructive testing model of taste components requires a large number of physical and chemical experiments to provide modeling reference values, which is not conducive to the rapid construction and efficient maintenance of the model.

### SUMMARY

In order to overcome the shortcomings of the existing technical solutions, the present disclosure quickly senses the seasoning taste of grains of rice and side dishes in finished fried rice in a non-destructive manner according to characteristics that a hyperspectral image signal is sensitive to the content of seasoning of fried rice, and provides a rapid and non-destructive quantitative evaluation method for taste characteristics of fried rice.

An objective of the present disclosure is to provide a rapid and non-destructive quantitative evaluation method for taste characteristics of fried rice, and includes three steps: constructing a quantitative detection model of seasoning for the fried rice, constructing a model for identifying types of materials of the fried rice, and performing quantitative characterization of the taste characteristics of the fried rice.

Step I, constructing a quantitative model of seasoning for the fried rice, including the following processes:

process I, using m kinds of seasoning liquid A_1, A_2, . . . , A_(m-1), and A_m as the seasoning for cooking the fried rice, and using n kinds of side dishes B_1, B_2, . . . , B_(n-1), and B_n and rice D as food ingredients for cooking the fried rice, where i-th seasoning liquid A_i has a standard concentration of C_A_i, the j-th side dish B_j has an average surface area of a single grain of S_B_j, the rice D has an average surface area of a single grain of S_D, C_A_i, S_B_j, and S_D are all positive numbers, m and n are both integers greater than 0, i∈[1, m], and j∈[1, n];

process II, taking e food ingredient combinations for the fried rice respectively, where each of the e food ingredient combinations for the fried rice contains N_B_j pieces of the j-th side dish B_j and N_D grains of the rice D; and taking e seasoning liquid combinations for the fried rice respectively, where each of the e seasoning liquid combinations for the fried rice contains the i-th seasoning liquid A_i with a volume of V_A_i ml, the A_i in the k-th seasoning liquid combination for the fried rice has the concentration of C_k_A_i=k*(C_A_i)/e, e is an integer greater than 2, k∈[1, e], N_B_j and N_D are both positive integers, and V_A_i is a positive number;

process III, according to an order of the concentrations of the seasoning liquid from low to high, cooking the fried rice with the e seasoning liquid combinations for the fried rice and the e food ingredient combinations for the fried rice by matching 1 seasoning liquid combination for the fried rice with 1 food ingredient combination for the fried rice to obtain e finished fried rice, where k-th finished fried rice contains an ingredient B_j&A_O&C_k of fried rice cooked with the side dish B_j and m kinds of the seasoning liquid A_i with the concentration of C_k_A_i, and an ingredient D&A_0&C_k of fried rice cooked with the rice D and the m kinds of the seasoning liquid A_i with the concentration of C_k_A_i;

process IV, performing hyperspectral image acquisition and spectral characteristic extraction:

taking i∈[1, m], j∈[1, n], and k∈[1, e], and taking f1 grains of ingredients B_j&A_0&C_k and D&A_0&C_k of the fried rice respectively for hyperspectral image acquisition and extraction of spectral characteristic variables to obtain a characteristic variable G1_A_i of the i-th seasoning A_i in the cooked fried rice; and extracting a sum of characteristic values Sum_g1_A_i_k of the seasoning A_i in the k-th finished fried rice respectively according to the characteristic variable G1_A_i, where f1 is a positive integer; and

process V, according to the sum of characteristic values Sum_g1_A_i_k of the seasoning A_i and a total amount (V_A_i)*k*(C_A_i)/e of the seasoning A_i in the k-th finished fried rice, assuming the quantitative model of the seasoning A_i to be y=F1_i(x)=x*h_A_i+b_A_i by using unknown numbers h_A_i and b_A_i, and since a total amount of the seasoning A_i in the k-th finished fried rice calculated using the model in combination with the sum of characteristic values Sum_g1_A_i_k of the seasoning A_i is equal to the total amount (V_A_i)*k*(C_A_i)/e of the A_i added when the k-th fried rice is cooked in process II of this step, establishing an equation (Sum_g1_A_i_k)*(h_A_i)+b_A_i=(V_A_i)*k*(C_A_i)/e for solving the unknown numbers h_A_i and b_A_i; and when the value of k is 1, 2 . . . , e-1, and e in turn, solving the unknown numbers h_A_i and b_A_i using the obtained equation so as to obtain the quantitative model of the seasoning A_i without unknown numbers as y=F1_i(x)=x*h_A_i+b_A_i, where the model y is the concentration (the amount of the seasoning per unit surface area) of the seasoning A_i, and x is the characteristic variable G1_A_i of the seasoning A_i.

Further, a process of extracting the G1_A_i in step I includes: taking each ingredient grain of the fried rice as a region of interest, and taking an average spectrum of each region of interest as spectral data of the sample to obtain full-band spectral information of the fried rice; and extracting the spectral characteristic variables using a principal component analysis (PCA) algorithm to obtain the characteristic variable G1_A_i of the i-th seasoning A_i in the cooked fried rice.

Further, a process of extracting the sum of characteristic values Sum_g1_A_i_k of the seasoning A_i in the k-th finished fried rice in step I includes: (1) extracting an average spectrum corresponding to f1 grains of the ingredient B_j&A_0&C_k of the fried rice added with the m kinds of seasoning liquid A_i with the concentration of C_k_A_i=k*(C_A_i)/e, and obtaining an average characteristic value g1_B_j&A_i&C_k corresponding to the B_j&A_0&C_k according to the characteristic variable G1_A_i of the seasoning A_i; and extracting an average spectrum corresponding to f1 grains of the ingredient D&A_0&C_k of the fried rice added with the m kinds of seasoning liquid A_i with the concentration of C_k_A_i=k*(C_A_i)/e, and obtaining an average characteristic value g1_D&A_i&C_k corresponding to the D&A_0&C_k according to the characteristic variable G1_A_i of the seasoning A_i; and (2) obtaining the sum of characteristic values of the seasoning A_i in the k-th finished fried rice Sum_g1_A_i_k=Σⱼ₌₁ⁿ(g1_B_j&A_i&C_k*S_B_j*N_B_j)+g1_D&A_i&C_k*S_D*N_D according to the number of grains N_B_j of the side dish B_j in the k-th fried rice and the average surface area of a single grain S_B_j, and the number of grains N_D of the rice D and the average surface area of a single grain S_D.

Step II, constructing a model for identifying types of materials of the fried rice, including the following processes:

process i, taking i∈[1, m] and j∈[1, n], taking f2 grains of ingredients B_j&A_0&C_e and D&A_0&C_e of the fried rice from e-th fried rice cooked in process III of step I respectively and dividing the ingredients of the fried rice randomly into a calibration set and a prediction set according to a ratio of d:1, performing hyperspectral image acquisition and extraction of a spectral characteristic variable G2_B&D of the types of the materials, and extracting a spectral characteristic value g2_B_j_cal corresponding to the side dish B_j and a spectral characteristic value g2_D_cal corresponding to the rice D in the calibration set, and a spectral characteristic value g2_B_j_pre corresponding to the side dish B_j and a spectral characteristic value g2_D_pre corresponding to the rice D in the prediction set respectively according to the spectral characteristic variable G2_B&D, where d and the f2 are positive integers; and

process ii, establishing the model for identifying the types of the materials of the fried rice Y=F2(X) in combination with a chemometric method by using the spectral characteristic variable G2_B&D as an independent variable X and the types of the materials of the fried rice as a dependent variable Y (using a reference value 0 to represent the rice D, and a reference value j to represent the side dish B_j).

Further, a method for extracting the G2_B&D in step II includes: taking each ingredient grain B_j&A_0&C_e and D&A_0&C_e of the fried rice as a region of interest, and taking an average spectrum of each region of interest as spectral data of the sample to obtain full-band spectral information of the fried rice sample; and screening to obtain a reflection strength corresponding to t characteristic wavelengths λ characterizing the types of the materials as the characteristic variable G2_B&D using a successive projections algorithm (SPA).

Further, the g2_B_j_cal in step II is an h1×t spectral characteristic value matrix composed of a reflection strength of h1 grains of B_j&A_0&C_e in the calibration set under characteristic wavelengths λ (there are t characteristic wavelengths); and

the g2_D_cal is an h1×t spectral characteristic value matrix composed of a reflection strength of h1 grains of rice D&A_0&C_e in the calibration set under the characteristic wavelengths λ, where there are t characteristic wavelengths; and the g2_B_j_pre and the g2_D_pre are h1*1/d×t spectral characteristic value matrices composed of reflection strengths of grains of the corresponding fried rice sample in the prediction set under the characteristic wavelengths λ.

Further, the chemometric method in step II is a support vector machine (SVM).

Step III, performing quantitative characterization of the taste characteristics of the fried rice, including the following processes:

process 1, using the m kinds of seasoning liquid A_1, A_2, A_(m-1), and A_m in process I of step I as the seasoning for cooking the fried rice, and using the n kinds of side dishes B_1, B_j, B_2, B_(n-1), and B_n and the rice D as the food ingredients for cooking the fried rice, where the i-th seasoning liquid A_i has the concentration of C′_A_i, the j-th side dish B_j has the average surface area of a single grain of S′_B_j, the rice D has the average surface area of a single grain of S′_D, and C′_A_i, S′_B_j, and S′_D are all positive numbers;

process 2, cooking the fried rice with the m kinds of seasoning A_i with a volume of V′_ A_i respectively, the n kinds of side dishes B_j with a number of grains of N′_B_j respectively, and the rice D with a number of grains of N′_D according to a cooking process in process III of step I; dispersing the cooked fried rice and spreading the fried rice into grains separated from each other to obtain N′=Σⱼ₌₁ⁿ(N′_B_j)+N′_D grains of the fried rice; performing hyperspectral image acquisition according to a method in process IV of step I, and obtaining a spectral characteristic value g1′_A_i_p of the seasoning A_i corresponding to the p-th grain in the fried rice according to the characteristic variable G1_A_i of the seasoning A_i; and obtaining a spectral characteristic value g2′_B&D_p for type identification corresponding to the p-th grain in the fried rice according to the spectral characteristic variable G2_B&D of the types of the materials in process i of step II, where p ∈ [1, N′];

process 3, setting a variable R_B_j to record the number of grains successfully identified for the side dish B_j in this step, and setting a variable R_D to record the number of grains successfully identified for the rice D in this step, where initial values of the R_B_j and the R_D are set to 0; and the value of p is 1, 2, . . . , N′-1, and N′ in turn:

firstly, substituting the spectral characteristic value g2′_B&D_p for type identification of the p-th grain into the model for identifying the types of the materials of the fried rice Y=F2(X) to obtain a type Yp of the material of the fried rice of the p-th grain;

secondly, taking the value of i as 1, 2, . . . , m-1, and m in turn, where Yp=0 indicates that the p-th grain is identified as the rice D, and the number of grains successfully identified R_D for the rice increases by 1; and substituting the spectral characteristic value g1′_A_i_p corresponding to the p-th grain into the quantitative model of the seasoning A_i y=F1_i(x) to obtain a relative content y1&D&R_D&A_i of the seasoning A_i corresponding to the (R_D)-th grain of the rice, and obtaining an absolute content y2&D&R_D&A_i=(y1&D&R_D&A_i)*S′_D of the seasoning A_i corresponding to the grain according to the surface area of the single grain of the rice D of S′_D, where

Yp=j indicates that the p-th grain is identified as the side dish B_j, and the number of grains successfully identified R_B_j for the side dish B_j increases by 1; and substituting the spectral characteristic value g1′_A_i_p corresponding to the p-th grain into the quantitative model of the seasoning A_i y=F1_i(x) to obtain a relative content y1&B_j&R_B_j&A_i of the seasoning A_i corresponding to the (R_B_j)-th grain of the side dish B_j, and obtaining an absolute content y2&B_j&R_B_j&A_i=(y1&B_j&R_B_j&A_i)*S′_B_j of the seasoning A_i corresponding to the grain according to the surface area of the single grain of the side dish B_j of S′_B_j; and

finally, obtaining a relative content y1&B_j&Uj&A_i and an absolute content y2&B_j&Uj&A_i of the seasoning A_i corresponding to a grain Uj of N′_B_j pieces of the side dish B_j in the fried rice in this step, and a relative content y1&D&VD&A_i and an absolute content y2&D&VD&A_i of the seasoning A_i corresponding to a grain VD of N′_D grains of the rice D, where Uj∈[1, N′_B_j], and VD∈[1, N′_D];

process 4, calculating relative taste characteristic evaluation indices, absolute taste characteristic evaluation indices, and taste uniformity characteristic evaluation indices of the taste characteristics of the fried rice, including the following specific processes:

(1) calculating the relative taste evaluation indices of the seasoning A_i on the side dish B_j and the rice D by the following method:

the relative taste evaluation index of the i-th seasoning A_i on the j-th side dish B_j as Cs_B_j&A_i=(Σ_{Uj=1}^{N′_B_j}y1&B_j&Uj&A_i)/N′_B_j, used to indicate an absorption capacity of the side dish B_j to the seasoning A_i; and

the relative taste evaluation index of the i-th seasoning A_i on the rice D as Cs_D&A_i=(Σ_{VD=1}^{N′_D}y1&D&VD &A_i)/N′_D, used to indicate an absorption capacity of the rice D to the seasoning A_i;

(2) calculating the absolute taste evaluation indices of the seasoning A_i on the side dish B_j and the rice D by the following method:

the absolute taste evaluation index of the i-th seasoning A_i on the j-th side dish B_j as Cd_B_j&A_i=(Σ_{VD=1}^{N′_D}y2&B_j&Uj&A_i)/N′_B_j, used to indicate total absorption of the single-grain side dish B_j to the seasoning A_i; and

the absolute taste evaluation index of the i-th seasoning A_i on the rice D as Cd_D&A_i=(Σ_{VD=1}^{N′_D}y2&D&VD&A_i)/N′_D, used to indicate total absorption of the single-grain rice D to the seasoning A_i; and

(3) calculating the taste uniformity evaluation indices of the seasoning A_i in grains of the side dish B_j and the rice D by the following method:

the taste uniformity evaluation index of the i-th seasoning A_i on the j-th side dish B_j as

$$σ1\_B \_j \& A\_i = \sqrt{\frac{{\sum }_{Uj = 1}^{{N}^{′} \_B \_j} {( y 2 \& B\_j \& Uj \& A\_i - Cd\_B \_j \& A\_i )}^{2}}{{N}^{′} \_B \_j}} ,$$

indicating a degree of difference in the content of the seasoning A_i among different grains of the side dish B_j;

the taste uniformity evaluation index of the i-th seasoning A_i on the rice D as

$$σ1\_D \& A\_i = \sqrt{\frac{{\sum }_{VD = 1}^{{N}^{′} \_D} {( y 2 \& D \& VD \& A\_i - Cd\_D \& A\_i )}^{2}}{{N}^{′} \_D}} ,$$

indicating a degree of difference in the content of the seasoning A_i among different grains of the rice D; and

the taste uniformity evaluation index of the i-th seasoning A_i in different types of food ingredients for the fried rice as

$$σ2\_A \_i = \sqrt{\frac{{\sum }_{j = 1}^{n} {( Cd\_B \_j \& A\_i - M\_Cd \_A \_i )}^{2} + {( Cd\_D \& A\_i - M\_Cd \_A \_i )}^{2}}{( n + 1 )}} ,$$

indicating a degree of difference in an average content of the seasoning A_i among different types of food ingredients, where

$$M\_Cd \_A \_i = \frac{{Σ}_{j = 1}^{n} Cd\_B \_j \& A\_i + Cd\_D \& A\_i}{( n + 1 )} ;$$

and

process 5, comparing the taste characteristic evaluation indices obtained in process 4 with standard indices of a standard sample to evaluate taste quality of the fried rice.

The standard sample refers to high-quality fried rice with perfect combination of color, aroma, and taste identified by combining sensory evaluation with physical and chemical analysis methods. At the same time, the standard sample can be adjusted according to the tastes of different places, and subdivided into different local standard samples to suit the local taste habits.

The present disclosure has the following beneficial effects:

In the present disclosure, the quantitative model of the seasoning is established based on sensitivity of a spectral signal to changes in the content of seasoning liquid. An analytic equation of key parameters of the quantitative model of the seasoning is established according to characteristics that the total amount of the seasoning in finished fried rice is equal to the total amount of the seasoning added during frying of the fried rice. The quantitative model of the seasoning is rapidly constructed by solving the equations. At the same time, the present disclosure quickly detects the content of different kinds of seasoning on single-grain materials of the fried rice by acquiring spectral characteristics of grains of the fried rice one by one in combination with the constructed quantitative model of the seasoning and the model for identifying the types of the materials of the fried rice, and hereby provides quantitative evaluation indices of the taste characteristics of the fried rice and a calculation method of the indices, so as to provide new technical means for studying and optimizing the taste characteristics of the fried rice.

### DETAILED DESCRIPTION OF THE EMBODIMENTS

The present disclosure is further described in detail below with reference to some specific embodiments, but the protection scope of the present disclosure is not limited thereto.

### Embodiment 1

A rapid and non-destructive quantitative evaluation method for taste characteristics of fried rice includes three steps: a quantitative model of seasoning for the fried rice is constructed, a model for identifying types of materials of the fried rice is constructed, and quantitative characterization of the taste characteristics of the fried rice is performed.

Step I: A quantitative model of seasoning for the fried rice is constructed, including the following processes.

Process I: Assuming m=2, n=2, C_A_1=90%, C_A_2=60%, S_B_1=6 cm², S_B_2=6 cm², and S_D=0.5 cm², soy sauce seasoning liquid A_1 and curry seasoning liquid A_2 are used as the seasonings for cooking the fried rice, and sausage B_1, carrot B_2, and rice D are used as food ingredients for cooking the fried rice. The soy sauce seasoning liquid A_1 has a standard concentration of 90% (volume percentage concentration), the curry seasoning liquid A_2 has a standard concentration of 60% (volume percentage concentration), the sausage B_1 has an average surface area of a single grain of 6 cm², the carrot B_2 has an average surface area of a single grain of 6 cm², and the rice D has an average surface area of a single grain of 0.5 cm².

Process II: Assuming e=3, V_A_1=10 ml, V_A_2=10 ml, N_B_1=50, N_B_2=50, and N_D=1800, 3 food ingredient combinations for the fried rice are taken respectively. Each of the 3 food ingredient combinations for the fried rice contains 50 pieces of the sausage B_1, 50 pieces of the carrot B_2, and 1800 grains of the rice D. 3 seasoning liquid combinations are taken respectively. The first seasoning liquid combination contains 10 ml of the soy sauce seasoning liquid A_1 with a concentration of C_1_A_1=30% and 10 ml of the curry seasoning liquid A_2 with a concentration C_1_A_2=20%. The second seasoning liquid combination contains 10 ml of the soy sauce seasoning liquid A_1 with a concentration of C_2_A_1=60% and 10 ml of the curry seasoning liquid A_2 with a concentration C_2_A_2=40%. The third seasoning liquid combination contains 10 ml of the soy sauce seasoning liquid A_1 with a concentration of C_3_A_1=90% and 10 ml of the curry seasoning liquid A_2 with a concentration C_3_A_2=60%.

Process III: According to an order of the concentrations of the seasoning liquid from low to high, the fried rice is cooked with the 3 food ingredient combinations for the fried rice and the 3 seasoning liquid combinations for the fried rice by matching 1 food ingredient combination for the fried rice with 1 seasoning liquid combination for the fried rice to obtain 3 finished fried rice. The 1st fried rice contains ingredients B_1&A_0&C_1, B_2&A_0&C_1, and D&A_0&C_1 of fried rice cooked with the sausage B_1, the carrot B_2, the rice D, the soy sauce seasoning liquid A_1 with a concentration of C_1_A_1=30%, and the curry seasoning liquid A_2 with a concentration C_1_A_2=20%. The 2nd fried rice contains ingredients B_1&A_0&C_2, B_2&A_0&C_2, and D&A_0&C_2 of fried rice cooked with the sausage B_1, the carrot B_2, the rice D, the soy sauce seasoning liquid A_1 with a concentration of C_2_A_1=60%, and the curry seasoning liquid A_2 with a concentration C_2_A_2=40%. The 3rd fried rice contains ingredients B_1&A_0&C_3, B_2&A_0&C_3, and D&A_0&C_3 of fried rice cooked with the sausage B_1, the carrot B_2, the rice D, the soy sauce seasoning liquid A_1 with a concentration of C_3_A_1=90%, and the curry seasoning liquid A_2 with a concentration C_3_A_2=60%.

Process IV: Hyperspectral image acquisition and spectral characteristic extraction are performed.

20 grains of ingredients B_1&A_0&C_1, B_2&A_0&C_1, D&A 0&C 1, B_1&A_0&C_2, B_2&A_0&C_2, D&A_0&C_2, B_1&A_0&C_3, B_2&A_0&C_3, and D&A_0&C_3 of the fried rice are taken respectively for hyperspectral image acquisition. Each ingredient grain of the fried rice is taken as a region of interest, and an average spectrum of each region of interest is taken as spectral data of the sample to obtain full-band spectral information of the fried rice. Spectral characteristic variables are extracted using a PCA algorithm to obtain a characteristic variable G1_A_1 of the soy sauce seasoning A_1 and a characteristic variable G1_A_2 of the curry seasoning A_2 in the cooked fried rice. Sums of characteristic values Sum_g1_A_1_1, Sum_g1_A_1_2, and Sum_g1_A_1_3 corresponding to the soy sauce seasoning A_1 at the concentration of 30%, 60%, and 90% in the 3 finished fried rice are extracted respectively according to the characteristic variable G1_A_1. Sums of characteristic values Sum_g1_A_2_1, Sum_g1_A_2_2, and Sum_g1_A_2_3 corresponding to the curry seasoning A_2 at the concentration of 20%, 40%, and 60% in the 3 finished fried rice are extracted respectively according to the characteristic variable G1_A_2.

A process of extracting the sum of characteristic values Sum_g1_A_i_k (i=1, 2; and k=1, 2, and 3) of the seasoning A_i in the k-th finished fried rice includes: (1) an average spectrum of 20 grains of the sausage B_1&A_0&C_k, an average spectrum of 20 grains of the carrot B_1&A_0&C_k, and an average spectrum of 20 grains of the rice D&A_0&C_k are extracted according to the spectral data obtained in process IV, and characteristic values g1_B_1&A_i&C_k, g1_B_2&A_i&C_k, and g1_D&A_i&C_k corresponding to the characteristic variable G1_A_i of the ingredients B_1&A_0&C_k, B_2&A_0&C_k, and D&A_0&C_k of the fried rice are extracted respectively according to the characteristic variable G1_A_i of the seasoning A_i in the cooked fried rice obtained in process IV; and (2) n=2, S_B_1=6 cm², S_B_2=6 cm², S_D=0.5 cm², N_B_1=50, N_B_2=50, and N_D=1800 are substituted into Sum_g1_A_i_k=Σⱼ₌₁ⁿ(g1_B_j&A_i&C_k*S_B_j*N_B_j)+g1_D&A_i&C_k*S_D*N_D to obtain Sum_g1_A_i_k=300*g1_B_1&A_i&C_k+300*g1_B_2&A_i&C_k+900*g1_D&A_i&C_k.

Process V: According to the sum of characteristic values Sum_g1_A_1_k of the soy sauce seasoning A_1 and a total amount of the soy sauce seasoning A_1 (V_A_1)*k*(C_A_1)/e=3*k ml in the k-th finished fried rice, the quantitative model of the soy sauce seasoning A_1 is assumed to be y=F1_1(x)=x*h_A_1+b_A_1 by using unknown numbers h_A_1 and b_A_1, and since a total amount of the soy sauce seasoning A_1 in the k-th fried rice calculated using the model in combination with the sum of characteristic values Sum_g1_A_1_k of the soy sauce seasoning A_1 is equal to the total amount of the soy sauce seasoning A_1 added when the k-th fried rice is cooked in process II of this step, an equation (Sum_g1_A_1_k)*(h_A_1)+b_A_1=3*k for solving the unknown numbers h_A_1 and b_A_1 is established. When the value of k is 1, 2, and 3 in turn, the unknown numbers h_A_1 and b_A 1 are solved using the obtained equation so as to obtain the quantitative model of the soy sauce seasoning A_1 without unknown numbers as y=F1_1(x)=x*h_A_1+b_A_1. The model y is the concentration (the amount of the seasoning per unit surface area) of the soy sauce seasoning A_1, and xis the characteristic variable G1_A_1 of the soy sauce seasoning A_1.

According to the sum of characteristic values Sum_g1_A_2_k of the curry seasoning A_2 and a total amount of the curry seasoning A_2 (V_A_2)*k*(C_A_2)/e=2*k ml in the k-th finished fried rice, the quantitative model of the curry seasoning A_2 is assumed to be y=F1_2(x)=x*h_A_2+b_A_2 by using unknown numbers h_A_2 and b_A_2, and since a total amount of the curry seasoning A_2 in the k-th fried rice calculated using the model in combination with the sum of characteristic values Sum_g1_A_2_k of the curry seasoning A_2 is equal to the total amount of the curry seasoning A_2 added when the k-th fried rice is cooked in process II of this step, an equation (Sum_g1_A_2_k)*(h_A_2)+b_A_2=2*k for solving the unknown numbers h_A_2 and b_A_2 is established. When the value of k is 1, 2, and 3 in turn, the unknown numbers h_A_2 and b_A_2 are solved using the obtained equation so as to obtain the quantitative model of the curry seasoning A_2 without unknown numbers as y=F1_2(x)=x*h_A_2+b_A_2. The model y is the concentration (the amount of the seasoning per unit surface area) of the curry seasoning A_2, and x is the characteristic variable G1_A_2 of the curry seasoning A_2.

Step II: A model for identifying types of materials of the fried rice is constructed, including the following processes.

Process i: 40 grains of ingredients B_1&A_0&C_3, B_2&A_0&C_3, and D&A_0&C_3 of the fried rice are taken from the third fried rice cooked in process III of step I respectively and divided randomly into a calibration set and a prediction set according to a ratio of 3:1, such that the calibration set contains 30 grains of the sausage B_1&A_0&C_3, 30 grains of the carrot B_2&A_0&C_3, and 30 grains of the rice D&A_0&C_3, and the prediction set contains 10 grains of the sausage Bi&A_0&C_3, 10 grains of the carrot B_2&A_0&C_3, and 10 grains of the rice D&A_0&C_3. Hyperspectral image acquisition is performed. Each ingredient grain B_1&A_0&C_3, B_2&A_0&C_3, and D&A_0&C_3 of the fried rice is taken as a region of interest, and an average spectrum of each region of interest is taken as spectral data of the sample to obtain full-band spectral information of the fried rice sample.

Screening is performed to obtain a reflection strength corresponding to t characteristic wavelengths λ characterizing the types of the materials as the characteristic variable G2_B&D using a SPA. A reflection strength of the grain of the sausage B_1&A_0&C_3 in the calibration set under characteristic wavelengths λ (there are t characteristic wavelengths) is extracted as a 30×t spectral characteristic value matrix g2_B_1_cal, a reflection strength of the grain of the carrot B_2&A_0&C_3 under characteristic wavelengths λ (there are t characteristic wavelengths) is extracted as a 30×t spectral characteristic value matrix g2_B_2_cal, and a reflection strength of the grain of the rice D&A_0&C_3 under characteristic wavelengths λ (there are t characteristic wavelengths) is extracted as a 30×t spectral characteristic value matrix g2_D_cal. Similarly, reflection strengths of grains of the corresponding fried rice sample in the prediction set under the characteristic wavelengths λ are extracted to obtain 10×t spectral characteristic value matrices g2_B_1_pre, g2_B_2_pre, and g2_D_pre.

Process ii: The model for identifying the types of the materials of the fried rice Y=F2(X) is established in combination with SVM by using the spectral characteristic variable G2_B&D as an independent variable X and the types of the materials of the fried rice as a dependent variable Y (using a reference value 0 to represent the rice D, a reference value 1 to represent the sausage B_1, and a reference value 2 to represent the carrot B_2).

Step III: Quantitative characterization of the taste characteristics of the fried rice is performed, including the following processes.

Process 1: C′_A_1=50%, C′_A_2=40%, S′_B_1=5 cm², S′_B_2=5 cm², and S′_D=0.6 cm². The soy sauce seasoning liquid A_1 and the curry seasoning liquid A_2 in process I of step I are used as the seasoning for cooking the fried rice, and the sausage B_1, the carrot B_2, and the rice D are used as the food ingredients for cooking the fried rice. The soy sauce seasoning liquid A_1 has a concentration of 50%, and the curry seasoning liquid A_2 has a concentration of 40%. The sausage B_1 and the carrot B_2 with an average surface area of a single grain of 5 cm², and the rice D with an average surface area of a single grain of 0.6 cm² are selected by a color sorter.

Process 2: V′_A_1=V′_A_2=15 ml, N′_B_1=N′_B_2=10, and N′_D=900. The fried rice is cooked with 15 ml of the soy sauce seasoning liquid A_1, 15 ml of the curry seasoning liquid A_2, 10 grains of the sausage B_1, 10 grains of the carrot, and 900 grains of the rice D added at one time according to a cooking process in process III of step I. The cooked fried rice is dispersed and the fried rice is spread into grains separated from each other to obtain N′=920 grains of the fried rice. Hyperspectral image acquisition is performed according to a method in process IV of step I, a spectral characteristic value g1′_A_1_p of the soy sauce seasoning A_1 corresponding to the p-th grain in the fried rice is obtained according to the characteristic variable G1_A_1 of the soy sauce seasoning A_1, and a spectral characteristic value g1′_A_2_p of the curry seasoning A_2 is obtained according to the characteristic variable G1_A_2 of the curry seasoning A_2. A spectral characteristic value g2′_B&D_p for type identification corresponding to the p-th grain in the fried rice is obtained according to the spectral characteristic variable G2_B&D of the types of the materials in process i of step II. p ∈ [1, 920].

Process 3: A variable R_B_1 is set to record the number of grains successfully identified for the sausage Bi in this step, a variable R_B_2 is set to record the number of grains successfully identified for the carrot B_2 in this step, and a variable R_D is set to record the number of grains successfully identified for the rice D in this step. Initial values of the R_B_1, the R_B_2, and the R_D are set to 0. The value of p is 1, 2, . . . , 919, and 920 in turn.

Firstly, the spectral characteristic value g2′_B&D_p for type identification of the p-th grain is substituted into the model for identifying the types of the materials of the fried rice Y=F2(X) to obtain a type Yp of the material of the fried rice of the p-th grain.

Secondly, Yp=0 indicates that the p-th grain is identified as the rice D, and the number of grains successfully identified R_D for the rice increases by 1. The spectral characteristic value g1′_A_1_p corresponding to the p-th grain is substituted into the quantitative model of the soy sauce seasoning A_1 as y=F1_1(x) to obtain a relative content y1&D&R_D&A_1 of the soy sauce seasoning A_1 corresponding to the (R_D)-th grain of the rice, the spectral characteristic value g1′_A_2_p corresponding to the p-th grain is substituted into the quantitative model of the curry seasoning A_2 y=F1_2(x) to obtain a relative content y1&D&R_D&A_2 of the curry seasoning A_2 corresponding to the (R_D)-th grain of the rice, and an absolute content y2&D&R_D&A_1=(y1&D&R_D&A_1)*0.6 of the soy sauce seasoning A_1 and an absolute content y2&D&R_D&A_2=(y1&D&R_D&A_2)*0.6 of the curry seasoning A_2 corresponding to the grain are obtained according to the surface area of the single grain of the rice D of S′_D=0.6 cm².

Yp=1 indicates that the p-th grain is identified as the sausage B_1, and the number of grains successfully identified R_B_1 for the sausage increases by 1. The spectral characteristic value g1′_A_1_p corresponding to the p-th grain is substituted into the quantitative model of the soy sauce seasoning A_1 as y=F1_1(x) to obtain a relative content y1&B_1&R_B_1&A_1 of the soy sauce seasoning A_1 corresponding to the (R_B_1)-th grain of the sausage, the spectral characteristic value g1′_A_2_ p corresponding to the p-th grain is substituted into the quantitative model of the curry seasoning A_2 y=F1_2(x) to obtain a relative content y1&B_1&R_B_1&A_2 of the curry seasoning A_2 corresponding to the (R_B_1)-th grain of the sausage, and an absolute content y2&B_1&R_B_1&A_1=(y1&B_1&R_B_1&A_1)*5 of the soy sauce seasoning A_1 and an absolute content y2&B_1&R_B_1&A_2=(y1&B_1&R_B_1&A_2)*5 of the curry seasoning A_2 corresponding to the grain are obtained according to the surface area of the single grain of the sausage B_1 of S′1_B_1=5 cm².

Yp=2 indicates that the p-th grain is identified as the carrot B_2, and the number of grains successfully identified R_B_2 for the carrot increases by 1. The spectral characteristic value g1′_A_1_p corresponding to the p-th grain is substituted into the quantitative model of the soy sauce seasoning A_1 as y=F1_1(x) to obtain a relative content y1&B_2&R_B_2&A_1 of the soy sauce seasoning A_1 corresponding to the (R_B_2)-th grain of the carrot, the spectral characteristic value g1′_A_2_p corresponding to the p-th grain is substituted into the quantitative model of the curry seasoning A_2 y=F1_2(x) to obtain a relative content y1&B_2&R_B_2&A_2 of the curry seasoning A_2 corresponding to the (R_B_2)-th grain of the carrot, and an absolute content y2&B_2&R_B_2&A_1=(y1&B_2&R_B_2&A_1)*5 of the soy sauce seasoning A_1 and an absolute content y2&B_2&R_B_2&A_2=(y1&B_2&R_B_2&A_2)*5 of the curry seasoning A_2 corresponding to the grain are obtained according to the surface area of the single grain of the carrot B_2 of S′_B_2=5 cm².

Finally, a relative content y1&B_1&U1&A_1 and an absolute content y2&B_1&U1&A_1 of the soy sauce seasoning A_1 and a relative content y1&B_1&U1 &A_2 and an absolute content y2&B_1&U1&A_2 of the curry seasoning A_2 corresponding to a grain U1 of the sausage B_1, a relative content y1&B_2&U2&A_1 and an absolute content y2&B_2&U2&A_1 of the soy sauce seasoning A_1 and a relative content y1&B_2&U2&A_2 and an absolute content y2&B_2&U2&A_2 of the curry seasoning A_2 corresponding to a grain U2 of the carrot B_2, and a relative content y1&D&VD&A_1 and an absolute content y2&D&VD&A_1 of the soy sauce seasoning A_1 and a relative content y1&D&VD&A_2 and an absolute content y2&D&VD&A_2 of the curry seasoning A_2 corresponding to a grain VD of the rice D in the fried rice in this step are obtained. U1 and U2∈E[1, 10], and VD∈[1, 900].

The relative content of the seasoning A_i corresponding to the grains of the fried rice is the concentration of the seasoning A_i corresponding to the grains of the fried rice (that is, the amount of the seasoning A_i per unit surface area), and the absolute content is the total amount of the seasoning A_i corresponding to the grains of the fried rice.

Process 4: Relative taste characteristic evaluation indices, absolute taste characteristic evaluation indices, and taste uniformity characteristic evaluation indices of the taste characteristics of the fried rice are calculated, including the following specific processes.

(1) The relative taste evaluation indices of the seasoning A_i on the sausage B_1, the carrot B_2, and the rice D are calculated by the following method:

the relative taste evaluation index of the soy sauce seasoning A_1 on the sausage B_1 as Cs_B_1&A_1=(Σ_{U1-1}¹⁰y1&B_1&U1&A_1)/10, used to indicate an absorption capacity of the sausage B_1 to the soy sauce seasoning A_1;

the relative taste evaluation index of the curry seasoning A_2 on the sausage B_1 as Cs_B_1&A_2=(Σn_{U1=1}¹⁰y1&B_1&U1&A_2)/10, used to indicate an absorption capacity of the sausage B_1 to the curry seasoning A_2;

the relative taste evaluation index of the soy sauce seasoning A_1 on the carrot B_2 as Cs_B_2&A_1=(Σ_{U2=1}¹⁰y1&B_2&U2&A_1)/10, used to indicate an absorption capacity of the carrot B_2 to the soy sauce seasoning A_1;

the relative taste evaluation index of the curry seasoning A_2 on the carrot B_2 as Cs_B_2&A_2=(Σ_{U2=1}¹⁰y1&B_2&U2&A_2)/10, used to indicate an absorption capacity of the carrot B_2 to the curry seasoning A_2;

the relative taste evaluation index of the soy sauce seasoning A_1 on the rice D as Cs_D&A_1=(Σ_{VD=1}⁹⁰⁰y1&D&VD&A_1)/900, used to indicate an absorption capacity of the rice D to the soy sauce seasoning A_1; and

the relative taste evaluation index of the curry seasoning A_2 on the rice D as Cs_D&A_2=(Σ_{VD=1}⁹⁰⁰y1&D&VD&A_2)/900, used to indicate an absorption capacity of the rice D to the curry seasoning A_2.

(2) The absolute taste evaluation indices of the seasoning A_i on the sausage B_1, the carrot B_2, and the rice D are calculated by the following method:

the absolute taste evaluation index of the soy sauce seasoning A_1 on the sausage B_1 as Cd_B_1&A_1=(Σ_{U1=1}¹⁰y2&B_1&U1&A_1)/10, used to indicate total absorption of the grains of the sausage B_1 to the soy sauce seasoning A_1;

the absolute taste evaluation index of the curry seasoning A_2 on the sausage B_1 as Cd_B_1&A_2=(Σ_{U1=1}¹⁰y2&B_1&U1&A_2)/10, used to indicate total absorption of the grains of the sausage B_1 to the curry seasoning A_2;

the absolute taste evaluation index of the soy sauce seasoning A_1 on the carrot B_2 as Cd_B_2&A_1=(Σ_{U2=1}¹⁰y2&B_2&U2&A_1)/10, used to indicate total absorption of the grains of the carrot B_2 to the soy sauce seasoning A_1;

the absolute taste evaluation index of the curry seasoning A_2 on the carrot B_2 as Cd_B_2&A_2=(Σ_{U2=1}¹⁰y2&B_2&U2&A_2)/10, used to indicate total absorption of the grains of the carrot B_2 to the curry seasoning A_2;

the absolute taste evaluation index of the soy sauce seasoning A_1 on the rice D as Cd_D&A_1=(Σ_{VD=1}⁹⁰⁰y2&D&VD&A_1)/900, used to indicate total absorption of the grains of the rice D to the soy sauce seasoning A_1; and

the absolute taste evaluation index of the curry seasoning A_2 on the rice D as Cd_D&A_2=(Σ_{VD=1}⁹⁰⁰y2&D&VD&A_2)/900, used to indicate total absorption of the grains of the rice D to the curry seasoning A_2.

(3) The taste uniformity evaluation indices of the seasoning A_i in the grains of the sausage B_1, the carrot B_2, and the rice D are calculated by the following method:

the taste uniformity evaluation index of the soy sauce seasoning A_1 on the sausage B_1 as

$$σ1\_B \_ 1 \& A\_ 1 = \sqrt{\frac{{Σ}_{U 1 = 1}^{10} {( y 2 \& B\_ 1 \& U 1 \& A\_ 1 - Cd\_B \_ 1 \& A\_ 1 )}^{2}}{10}} ,$$

indicating a degree of difference in the content of the soy sauce seasoning A_1 among different grains of the sausage B_1;

the taste uniformity evaluation index of the curry seasoning A_2 on the sausage B_1 as

$$σ1\_B \_ 1 \& A\_ 2 = \sqrt{\frac{{Σ}_{U 1 = 1}^{10} {( y 2 \& B\_ 1 \& U 1 \& A\_ 2 - Cd\_B \_ 1 \& A\_ 2 )}^{2}}{10}} ,$$

indicating a degree of difference in the content of the curry seasoning A_2 among different grains of the sausage B_1;

the taste uniformity evaluation index of the soy sauce seasoning A_1 on the carrot B_2 as

$$σ1\_B \_ 2 \& A\_ 1 = \sqrt{\frac{{Σ}_{U 2 = 1}^{10} {( y 2 \& B\_ 2 \& U 2 \& A\_ 1 - Cd\_B \_ 2 \& A\_ 1 )}^{2}}{10}} ,$$

indicating a degree of difference in the content of the soy sauce seasoning A_1 among different grains of the carrot B_2;

the taste uniformity evaluation index of the curry seasoning A_2 on the carrot B_2 as

$$σ1\_B \_ 2 \& A\_ 2 = \sqrt{\frac{{Σ}_{U 2 = 1}^{10} {( y 2 \& B\_ 2 \& U 2 \& A\_ 2 - Cd\_B \_ 2 \& A\_ 2 )}^{2}}{10}} ,$$

indicating a degree of difference in the content of the curry seasoning A_2 among different grains of the carrot B_2;

the taste uniformity evaluation index of the soy sauce seasoning A_1 on the rice D as

$$σ1\_D \& A\_ 1 = \sqrt{\frac{{Σ}_{VD = 1}^{900} {( y 2 \& D \& VD \& A\_ 1 - Cd\_D \& A\_ 1 )}^{2}}{900}} ,$$

indicating a degree of difference in the content of the soy sauce seasoning A_1 among different grains of the rice D;

the taste uniformity evaluation index of the curry seasoning A_2 on the rice D as

$$σ1\_D \& A\_ 2 = \sqrt{\frac{{Σ}_{VD = 1}^{900} {( y 2 \& D \& VD \& A\_ 2 - Cd\_D \& A\_ 2 )}^{2}}{900}} ,$$

indicating a degree of difference in the content of the curry seasoning A_2 among different grains of the rice D;

the taste uniformity evaluation index of the soy sauce seasoning A_1 in different types of food ingredients for the fried rice as

$$σ2\_A \_ 1 = \sqrt{\frac{\begin{matrix}\begin{matrix}{( Cd\_B \_ 1 \& A\_ 1 - M\_Cd \_A \_ 1 )}^{2} + \\ {( Cd\_B \_ 2 \& A\_ 1 - M\_Cd \_A \_ 1 )}^{2} +\end{matrix} \\ {( Cd\_D \& A\_ 1 - M\_Cd \_A \_ 1 )}^{2}\end{matrix}}{3}} ,$$

indicating a degree of difference in an average content of the soy sauce seasoning A_1 among different types of food ingredients, where

$$M\_Cd \_A \_ 1 = \frac{Cd\_B \_ 1 \& A\_ 1 + Cd\_B \_ 2 \& A\_ 1 + Cd\_D \& A\_ 1}{3} ;$$

and

the taste uniformity evaluation index of the curry seasoning A_2 in different types of food ingredients for the fried rice as

$$σ2\_A \_ 2 = \sqrt{\frac{\begin{matrix}\begin{matrix}{( Cd\_B \_ 1 \& A\_ 2 - M\_Cd \_A \_ 2 )}^{2} + \\ {( Cd\_B \_ 2 \& A\_ 2 - M\_Cd \_A \_ 2 )}^{2} +\end{matrix} \\ {( Cd\_D \& A\_ 2 - M\_Cd \_A \_ 2 )}^{2}\end{matrix}}{3}} ,$$

indicating a degree of difference in an average content of the curry seasoning A_2 among different types of food ingredients, where

$$M\_Cd \_A \_ 2 = \frac{Cd\_B \_ 1 \& A\_ 2 + Cd\_B \_ 2 \& A\_ 2 + Cd\_D \& A\_ 2}{3} .$$

Process 5: In the early stage, standard indices for taste evaluation of the fried rice are established according to a standard sample by sensory evaluation. The taste characteristic evaluation indices obtained in process 4 are compared with the standard indices. The evaluation indices closer to the standard indices indicate better taste quality of the fried rice. Within ±10% of the standard indices, the quality is considered to be excellent.

The standard sample refers to high-quality fried rice with perfect combination of color, aroma, and taste identified by combining sensory evaluation with physical and chemical analysis methods.

1. A rapid quantitative evaluation method for taste characteristics of fried rice, comprising the following steps:
step I, constructing a quantitative model of seasoning for the fried rice, comprising the following processes:
process I, using m kinds of seasoning liquid A_1, A_2, . . . , A_(m-1), and A_m as the seasoning for cooking the fried rice, and using n kinds of side dishes B_1, B_2, . . . , B_(n-1), and B_n and rice D as food ingredients for cooking the fried rice, wherein an i-th seasoning liquid A_i has a standard concentration of C_A_i, a j-th side dish B_j has an average surface area of a single grain of S_B_j, the rice D has an average surface area of a single grain of S_D, C_A_i, S_B_j, and S_D are all positive numbers, m and n are both integers greater than 0, i∈[1, m], and j∈[1, n];
process II, taking e food ingredient combinations for the fried rice respectively, wherein each of the e food ingredient combinations for the fried rice contains N_B_j pieces of the j-th side dish B_j and N_D grains of the rice D; and taking e seasoning liquid combinations for the fried rice respectively, wherein each of the e seasoning liquid combinations for the fried rice contains the i-th seasoning liquid A_i with a volume of V_A_i ml, the seasoning liquid A_i in a k-th seasoning liquid combination for the fried rice has a concentration of C_k_A_i=k*(C_A_i)/e, e is an integer greater than 2, k∈[1, e], N_B_j and N_D are both positive integers, and V_A_i is a positive number;
process III, according to an order of the concentrations of the seasoning liquid from low to high, cooking the fried rice with the e seasoning liquid combinations for the fried rice and the e food ingredient combinations for the fried rice by matching 1 seasoning liquid combination for the fried rice with 1 food ingredient combination for the fried rice to obtain e finished fried rice, wherein k-th finished fried rice contains an ingredient B_j&A_0&C_k of fried rice cooked with the side dish B_j and m kinds of the seasoning liquid A_i with the concentration of C_k_A_i, and an ingredient D&A_0&C_k of fried rice cooked with the rice D and the m kinds of the seasoning liquid A_i with the concentration of C_k_A_i;
process IV, performing hyperspectral image acquisition and spectral characteristic extraction:
taking i∈[1, m], j∈[1, n], and k∈[1, e], and taking f1 grains of ingredients B_j&A_0&C_k and D&A_0&C_k of the fried rice respectively for hyperspectral image acquisition and extraction of spectral characteristic variables to obtain a characteristic variable G1_A_i of the i-th seasoning A_i in the cooked fried rice; and extracting a sum of characteristic values Sum_g1_A_i_k of the seasoning A_i in the k-th finished fried rice respectively according to the characteristic variable G1_A_i, wherein f1 is a positive integer; and
process V, according to the sum of characteristic values Sum_g1_A_i_k of the seasoning A_i and a total amount (V_A_i)*k*(C_A_i)/e of the seasoning A_i in the k-th finished fried rice, assuming the quantitative model of the seasoning A_i to be y=F1_i(x)=x*h_A_i+b_A_i by using unknown numbers h_A_i and b_A_i, and since a total amount of the seasoning A_i in the k-th finished fried rice calculated using the model in combination with the sum of characteristic values Sum_g1_A_i_k of the seasoning A_i is equal to the total amount (V_A_i)*k*(C_A_i)/e of the A_i added when the k-th fried rice is cooked in process II of this step, establishing an equation (Sum_g1_A_i_k)*(h_A_i)+b_A_i=(V_A_i)*k*(C_A_i)/e for solving the unknown numbers h_A_i and b_A_i; and when the value of k is 1, 2 . . . , e-1, and e in turn, solving the unknown numbers h_A_i and b_A_i using the obtained equation so as to obtain the quantitative model of the seasoning A_i without unknown numbers as y=F1_i(x)=x*h_A_i+b_A_i, wherein y is the concentration of the seasoning A_i, and x is the characteristic variable G1_A_i of the seasoning A_i;
step II, constructing a model for identifying types of materials of the fried rice, comprising the following processes:
process i, taking i∈[1, m] and j∈[1, n], taking f2 grains of ingredients B_j&A_0&C_e and D&A_0&C_e of the fried rice from e-th fried rice cooked in process III of step I respectively and dividing the ingredients of the fried rice randomly into a calibration set and a prediction set according to a ratio of d:1, performing hyperspectral image acquisition and extraction of a spectral characteristic variable G2_B&D of the types of the materials, and extracting a spectral characteristic value g2_B_j cal corresponding to the side dish B_j and a spectral characteristic value g2_D_cal corresponding to the rice D in the calibration set, and a spectral characteristic value g2_B_j_pre corresponding to the side dish B_j and a spectral characteristic value g2_D_pre corresponding to the rice D in the prediction set respectively according to the spectral characteristic variable G2_B&D, wherein d and the f2 are positive integers; and
process ii, establishing the model for identifying the types of the materials of the fried rice Y=F2(X) in combination with a chemometric method by using the spectral characteristic variable G2_B&D as an independent variable X and the types of the materials of the fried rice as a dependent variable Y and using a reference value 0 to represent the rice D, and a reference value j to represent the side dish B_j; and
step III, performing quantitative characterization of the taste characteristics of the fried rice, comprising the following processes:
process 1, using the m kinds of seasoning liquid A_1, A_2, . . . , A (m-1), and A_m in process I of step I as the seasoning for cooking the fried rice, and using the n kinds of side dishes B_1, B_2, . . . , B (n-1), and B_n and the rice D as the food ingredients for cooking the fried rice, wherein the i-th seasoning liquid A_i has the concentration of C′_A_i, the j-th side dish B_j has the average surface area of a single grain of S′_B_j, the rice D has the average surface area of a single grain of S′_D, and C′_A_i, S′_B_j, and S′_D are all positive numbers;
process 2, cooking the fried rice with the m kinds of seasoning A_i with a volume of V′_A_i respectively, the n kinds of side dishes B_j with a number of grains of N′_B_j respectively, and the rice D with a number of grains of N′_D according to a cooking process in process III of step I; dispersing the cooked fried rice and spreading the fried rice into grains separated from each other to obtain N′=Σⱼ₌₁ⁿ(N′_B_j)+N′_D grains of the fried rice; performing hyperspectral image acquisition according to a method in process IV of step I, and obtaining a spectral characteristic value gr_A_i_p of the seasoning A_i corresponding to a p-th grain in the fried rice according to the characteristic variable G1_A_i of the seasoning A_i; and obtaining a spectral characteristic value g2′_B&D_p for type identification corresponding to the p-th grain in the fried rice according to the spectral characteristic variable G2_B&D of the types of the materials in process i of step II, wherein p∈[1,N′];
process 3, setting a variable R_B_j to record the number of grains successfully identified for the side dish B_j in this step, and setting a variable R_D to record the number of grains successfully identified for the rice D in this step, wherein initial values of the R_B_j and the R_D are set to 0; and the value of p is 1, 2, . . . , N′-1, and N′ in turn:
firstly, substituting the spectral characteristic value g2′_B&D_p for type identification of the p-th grain into the model for identifying the types of the materials of the fried rice Y=F2(X) to obtain a type Yp of the material of the fried rice of the p-th grain;
secondly, taking the value of i as 1, 2, . . . , m-1, and m in turn, wherein Yp=0 indicates that the p-th grain is identified as the rice D, and the number of grains successfully identified R_D for the rice increases by 1; and substituting the spectral characteristic value g1′_A_i_p corresponding to the p-th grain into the quantitative model of the seasoning A_i y=F1_i(x) to obtain a relative content y1&D&R_D&A_i of the seasoning A_i corresponding to a (R_D)-th grain of the rice, and obtaining an absolute content y2&D&R_D&A_i=(y1&D&R_D&A_S′_D of the seasoning A_i corresponding to the grain according to the surface area of the single grain of the rice D of S′_D, wherein
Yp=j indicates that the p-th grain is identified as the side dish B_j, and the number of grains successfully identified R_B_j for the side dish B_j increases by 1; and substituting the spectral characteristic value g1′_A_i_p corresponding to the p-th grain into the quantitative model of the seasoning A_i y=F1_i(x) to obtain a relative content y1&B_j&R_B_j&A_i of the seasoning A_i corresponding to a (R_B_j)-th grain of the side dish B_j, and obtaining an absolute content y2&B_j&R_B_j&A_i=(y1&B_j&R_B_j&A_i)*S′_B_j of the seasoning A_i corresponding to the grain according to the surface area of the single grain of the side dish B_j of S′_B_j; and
finally, obtaining a relative content y1&B_j&Uj&A_i and an absolute content y2&B_j&Uj&A_i of the seasoning A_i corresponding to a grain Uj of N′_B_j pieces of the side dish B_j in the fried rice in this step, and a relative content y1&D&VD&A_i and an absolute content y2&D&VD&A_i of the seasoning A_i corresponding to a grain VD of N′_D grains of the rice D, wherein Uj∈[1, N′_B_j], and VD∈[1, N′_D];
process 4, calculating relative taste characteristic evaluation indices, absolute taste characteristic evaluation indices, and taste uniformity characteristic evaluation indices of the taste characteristics of the fried rice, comprising the following specific processes:
(1) calculating the relative taste evaluation indices of the seasoning A_i on the side dish B_j and the rice D by the following method:
the relative taste evaluation index of the i-th seasoning A_i on the j-th side dish B_j as Cs_B_j&A_i=(Σ_{Uj=1}^{N′_B_j}y1&B_j&Uj&A_i)/N′_B_j, used to indicate an absorption capacity of the side dish B_j to the seasoning A_i; and
the relative taste evaluation index of the i-th seasoning A_i on the rice D as Cs_D&A_i=(Σ_{VD=1}^{N′_D}y1&D&VD&A_i)/N′_D, used to indicate an absorption capacity of the rice D to the seasoning A_i;
(2) calculating the absolute taste evaluation indices of the seasoning A_i on the side dish B_j and the rice D by the following method:
the absolute taste evaluation index of the i-th seasoning A_i on the j-th side dish B_j as Cd_B_j&A_i=(Σ_{Uj=1}^{N′B_j}y2&B_j&Uj&A_i)/N′_B_j, used to indicate total absorption of the single-grain side dish B_j to the seasoning A_i; and
the absolute taste evaluation index of the i-th seasoning A_i on the rice D as Cd_ D&A_i=(Σ_{VD=1}^{N′_D}y2&D&VD&A_i)/N′_D, used to indicate total absorption of the single-grain rice D to the seasoning A_i; and
(3) calculating the taste uniformity evaluation indices of the seasoning A_i in grains of the side dish B_j and the rice D by the following method:
the taste uniformity evaluation index of the i-th seasoning A_i on the j-th side dish B_j as
$$σ1\_B \_j \& A\_i = \sqrt{\frac{{\sum }_{Uj = 1}^{{N}^{′} \_ B \_ j} {( y 2 \& B\_j \& Uj \& A\_i - Cd\_B \_J \& A\_i )}^{2}}{{N}^{′} \_B \_j}} ,$$
indicating a degree of difference in a content of the seasoning A_i among different grains of the side dish B_j;
the taste uniformity evaluation index of the i-th seasoning A_i on the rice D as
$$σ1\_D \& A\_i = \sqrt{\frac{{\sum }_{VD = 1}^{{N}^{′} \_ D} {( y 2 \& D \& VD \& A\_i - Cd\_D \& A\_i )}^{2}}{{N}^{′} \_D}} ,$$
indicating a degree of difference in a content of the seasoning A_i among different grains of the rice D; and
the taste uniformity evaluation index of the i-th seasoning A_i in different types of food ingredients for the fried rice as
$$σ2\_A \_i = \sqrt{\frac{\begin{matrix}{\sum }_{j = 1}^{n} {( Cd\_B \_j \& A\_i - M\_Cd \_A \_i )}^{2} + \\ {( Cd\_D \& A\_i - M\_Cd \_A \_i )}^{2}\end{matrix}}{( n + 1 )}} ,$$
indicating a degree of difference in an average content of the seasoning A_i among different types of food ingredients, wherein
$$M\_Cd \_A \_i = \frac{{\sum }_{j = 1}^{n} Cd\_B \_j \& A\_i + Cd\_D \& A\_i}{( n + 1 )} [ [ . ] ] ;$$
and process 5, comparing the taste characteristic evaluation indices obtained in
process 4 with standard indices of a standard sample to evaluate taste quality of the fried rice.

2. The rapid quantitative evaluation method for taste characteristics of fried rice according to claim 1, wherein a process of extracting the G1_A_i in step I comprises: taking each ingredient grain of the fried rice as a region of interest, and taking an average spectrum of each region of interest as spectral data of the sample to obtain full-band spectral information of the fried rice; and
extracting the spectral characteristic variables using a principal component analysis algorithm to obtain the characteristic variable G1_A_i of the i-th seasoning A_i in the cooked fried rice.

3. The rapid quantitative evaluation method for taste characteristics of fried rice according to claim 1, wherein a process of extracting the sum of characteristic values Sum_g1_A_i_k of the seasoning A_i in the k-th finished fried rice in step I comprises:
(1) extracting an average spectrum corresponding to f1 grains of the ingredient B_j&A_0&C_k of the fried rice added with the m kinds of seasoning liquid A_i with the concentration of C_k_A_i=k*(C_A_i)/e, and obtaining an average characteristic value g1_B_j&A_i&C_k corresponding to the B_j&A_0&C_k according to the characteristic variable G1_A_i of the seasoning A_i; and extracting an average spectrum corresponding to f1 grains of the ingredient D&A_0&C_k of the fried rice added with the m kinds of seasoning liquid A_i with the concentration of C_k_A_i=k*(C_A_i)/e, and obtaining an average characteristic value g1_D&A_i&C_k corresponding to the D&A_0&C_k according to the characteristic variable G1_A_i of the seasoning A_i; and
(2) obtaining the sum of characteristic values of the seasoning A_i in the k-th finished fried rice Sum_g1_A_i_k=Σⱼ₌₁ⁿ(g1_B_j&A_i&C_k*S_B_j*N_B_j)+*S_D*N_D according to the number of grains N_B_j of the side dish B_j in the k-th fried rice and the average surface area of a single grain S_B_j, and the number of grains N_D of the rice D and the average surface area of a single grain S_D.

4. The rapid quantitative evaluation method for taste characteristics of fried rice according to claim 1, wherein a method for extracting the G2_B&D in step II comprises: taking each ingredient grain B_j&A_0&C_e and D&A_0&C_e of the fried rice as a region of interest, and taking an average spectrum of each region of interest as spectral data of the sample to obtain full-band spectral information of the fried rice sample; and screening to obtain a reflection strength corresponding to t characteristic wavelengths λ characterizing the types of the materials as the characteristic variable G2_B&D using a successive projections algorithm.

5. The rapid quantitative evaluation method for the taste characteristics of the fried rice according to claim 1, wherein the g2_B_j_cal in step II is an h1×t spectral characteristic value matrix composed of a reflection strength of h1 grains of B_j&A_0&C_e in the calibration set under characteristic wavelengths λ, wherein a number of the characteristic wavelengths is t; and
the g2_D_cal is an h1×t spectral characteristic value matrix composed of a reflection strength of h1 grains of rice D&A_0&C_e in the calibration set under the characteristic wavelengths λ, and the number of the characteristic wavelengths is t; and the g2_B_j_pre and the g2_D_pre are h1*1/d×t spectral characteristic value matrices composed of reflection strengths of grains of the corresponding fried rice sample in the prediction set under the characteristic wavelengths λ.

6. The rapid quantitative evaluation method for taste characteristics of fried rice according to claim 1, wherein the chemometric method in step II is a support vector machine.
